(19) United States
(12) Patent Application Publication
(10) Pub. No.: US 2011/0206747 A1
(43) Pub. Date: Aug. 25, 2011

(54) Multiplication of the efficacy of anti-infectious agents by a composition further comprising a dispersing agent together with a metal activating agent

(75) Inventors: Pierre LAGNY, Co. Kildare (IE); Jean-Jacques HERAULT, London (GB)

(73) Assignee: EAST COAST PHARMACEUTICAL RESEARCH LLC

(21) Appl. No.: 12/783,027

(22) Filed: May 19, 2010

(30) Foreign Application Priority Data
Feb. 23, 2010 (EP) 10305180.1

(51) Int. Cl. A61K 8/02; A61K 36/53; A61K 36/61; A61K 36/54; A61K 36/82; A61K 36/752; A61K 9/127; A61K 9/14; A61K 33/16; A61K 33/24; A01N 65/00; A01N 59/16; A61Q 90/00

(52) U.S. Cl. 424/401; 424/745; 424/742; 424/739; 424/729; 424/736; 424/450; 424/489; 424/673; 424/646; 424/650

(57) ABSTRACT

The present invention relates to a composition inhibiting or destroying at least one living or unicellular organism, which comprises at least one anti-infectious agent and at least one activating agent containing at least one metal element, together with at least one dispersing agent. The present invention also relates to the utilization of such a composition.

The present invention relates in general to the destruction and/or the inhibition of living or unicellular organisms such as protozoa, microbes, bacteria, gametes, fungi, yeasts, parasites or others. Many substances inhibiting or destroying unicellular living organisms are already known, and these include the surfactant agents such as quaternary ammonium salts. In particular, it is known that quaternary ammonium halides such as benzalkonium chloride (or alkyl-benzyl-dimethyl-ammonium chloride), alone or in combination with other active ingredients, are advantageous in these applications (see, for example, British Patents GB1 554 615, French Patents FR 2 431 859, FR 2 483 177, FR 2 379 508, FR 2 384 497, FR 2 457 641, FR 2 573 624, FR 2 418 221, FR 2 562 888, European Patents EP 0 243 713, EP 0 175 338, EP 0 132 963, EP 0 127 131, EP 0 094 562, EP 0 076 136, EP 0 068 399, EP 0 037 593, and international applications WO 84/00877 and WO 84/02649).

Moreover, the prior art has already provided many processes for manufacturing these quaternary ammonium salts (among quaternary ammonium halides), directly applicable to the chlorides and/or iodides, and/or bromides, and/or chloro-iodites (see above Patents and also French Patents FR 2 472 558, FR 2 033 044 and European Patents EP 0 094 552 and EP 0 012 296).

Ammonium fluoride and processes for preparing and purifying it are also known (see, for example, French Patents FR 2 244 713, 2 253 710 and European Patent EP 0 002 016), as are perfluorinated or polyfluorinated quaternary ammonium salts (for example, French Patents 2 038 421, 2 051 095, 2 153 489 and European Patents EP 0 073 760, EP 0 100 478, EP 0 100 477, and EP 0149 172).

Ionic fluorine is, moreover, well known for its anti caries properties in dental applications (for example, U.S. Pat. No. 4,473,547), optionally combined with a cationic quaternary ammonium compound (see French Patents 1 486 676 and 1 297 708). In these latter documents, the advantage of combining, in one and the same compound, on the one hand fluorine, known for its anti caries properties, and on the other hand, surfactant quaternary ammonium salts, known for their bactericidal properties, is demonstrated. However, no genuinely synergistic activity is demonstrated in these applications for dentifrices, with the quaternary ammonium fluorides tested, either with respect to anti caries properties or with respect to bactericidal properties.

French Patent FR 1 297 708 describes in particular lauryl-benzyl-dimethyl-ammonium fluoride, a process for preparing this fluoride and its application in a toothpaste.

Italian Patent FR 1 153 530 describes a process for preparing quaternary ammonium carbonates and then, from these carbonates, quaternary ammonium halides by anion exchange with the corresponding acid, which is stronger than carbonic acid. However, while the compounds obtained by means of this process are suitable for application in the disinfection of surfaces and in industry, they cannot be used directly in human beings or animals, for example in the pharmaceutical compositions in which the compounds must be of high purity.

In European Patent EP 0308564, the problem of providing a composition that completely inhibits or destroys unicellular living organisms and which is simultaneously applicable to living organisms, human beings, animals or plants, without thereby causing harmful side effects, is raised. The solution to this problem described in EP 0308564 consists in a composition containing at least a myristalkonium fluoride, which may be used in human beings or animals. However, this composition has the disadvantage of causing an irritation at the site of injection, except when it is used through central administration. This irritation, caused by the necessary amount of active ingredient in the composition, may be improved by the reduction of the amount of this active ingredient when it is combined with an activating agent and a dispersing agent.

Moreover, European Patent EP 0310476 describes a pharmaceutical composition containing ionic and ionizable fluorine, and ionic or ionizable lithium, in particular lithium fluoride. However, this composition has the disadvantage of containing no dispersing agent, and therefore of rendering more difficult the relationship between the composition and the infectious agent, and this is completely modified by the addition of the dispersing agent.

However the compositions described in the above-mentioned patents of the prior art require sufficiently great amounts of anti-infectious agents to be effective. But these great amounts have the major disadvantage of causing undesirable side effects in human beings and animals.

The applicant has discovered in a surprising manner that the action of one or several activating agents comprising at least one metal element (in particular in the form of ions, salts or metal colloids, either of nanoparticular type or not), together with dispersing agents which are liposomal, or micellar, or peptidic, or in the form of microemulsion or of nanoemulsion, or in the form of microcapsule, in a composition comprising an anti-infectious agent, allows an increase in its inhibiting or destroying potency towards living or unicellular organisms.

On the other hand, the combination as it is a combination of a dispersing agent with a metal activator has by itself no potency to inhibit or destroy an infection, and it only has a catalytic effect towards the anti-infectious agents used (also called active ingredients in the present application). This has for direct consequence that the amount necessary to inhibit or destroy living or unicellular organisms is considerably reduced in relation to that usually necessary in the absence of these catalytic compounds (metal activating agent(s) used together with a dispersing agent).

Moreover, the applicant has discovered that the metal element of the activating agent (or agents) must be present in the composition in an amount not exceeding 20 ppm or mg/L of composition. Indeed, doses superior to 20 ppm or mg/L cause in a surprising manner a reverse effect, i.e. a reduction of the inhibiting and/or destroying potency of the active ingredients.

More particularly, the present invention hence relates to a composition that inhibits and/or destroys at least one living or unicellular organism, and which comprises:
at least one anti-infectious agent, and
at least one activating agent containing at least one metal element.

According to the invention, the composition further comprises at least one combined (in the sense of together with) dispersing agent which is liposomal, or micellar, or peptidic, or in the form of microemulsion or of nanoemulsion, or in the form of microcapsule, and the metal element of the activating agent is present in said composition in an amount not exceeding 20 mg/L.

Dispersing Agent

The dispersing agents have been known and used for years but none of them has been used together with metal ions in order to increase the efficacy of an anti-infectious agent.

The activity of liposomal dispersing agents on anti-infectious agents is known in the art, as is attested by M. Ravaoarinoro's publications: 1. “Pharmacokinetics of cationic liposome-encapsuled doxycycline in mice challenged with genital infection by Chlamydia trachomatis” of Selliah S., Ravaoarinoro M, in Chemotherapy, 2004 April, 50 (1), 17-21. PMID: 15084800 [PubMed—indexed for Medline]; 2. “In vitro inhibition of Chlamydia trachomatis growth by liposome-encapsuled cyclines” of Sangaré L., Morisset R., Ravaoarinoro M, in Pathol Biol (Paris), 2001 February, 49 (1), 53-6. French, PMID: 11265224 [PubMed—indexed for Medline]; 3. “Effets of cationic liposome-encapsuled doxycycline on experimental Chlamydia trachomatis genital infection in mice.” of Sangaré L., Morisset R., Gaboury L., Ravaoarinoro M., in J Antimicrob Chemother., 2001 March, 47 (3), 323-31. PMID: 11222565 [PubMed—indexed for Medline]; 4. “In-vitro anti-chlamydial activities of free and liposomal tetracycline and doxycycline” of Sangaré L., Morisset R., Ravaoarinoro M., in J Med Microbiol., 1999 July, 48 (7), 689-93. PMID: 10403420 [PubMed—indexed for Medline]; 5. “Incorporation rates, stabilities, cytotoxicities and release of liposomal tetracycline and doxycycline in human serum” of Sangaré L., Morisset R., Omri A., Ravaoarinoro M., in J Antimicrob Chemother., 1998 December, 42 (6), 831-4.PMID: 10052911 [PubMed—indexed for Medline]; 6. “Comparison of the bactericidal action of amilkacin, netilmicin and tobramycin in free and liposomal formulation against Pseudomonas aeruginosa.”, of Omri A., Ravaoarinoro M., in Chemotherapy, 1996 May-June, 42 (3), 170-6. PMID: 8983883 [PubMed—indexed for Medline]; 7. “Incorporation, release and in-vitro antibacterial activity of liposomal aminoglycosides against Pseudomonas aeruginosa.”, of Omri A., Ravaoarinoro M., Poisson M., in J Antimicrob Chemother., 1995 October, 36 (4), 631-9. PMID: 8591937 [PubMed—indexed for Medline]; 8. “Liposomes, in the treatment of infections”, of Ravaoarinoro M., Toma E, in Ann Med Interne (Paris),1993, 144 (3), 182-7. Review. French. PMID: 8368703 [PubMed—indexed for Medline]; 9. “Efficient entrapment of amikacin and teicoplamin in liposomes.”, of Ravaoarinoro M., Toma E., Agbaba O., Morisset R., in J Drug Target., 1993, 1 (3), 191-5. PMID: 8069560 [PubMed—indexed for Medline].

However, in these publications, the liposomal dispersing agent is not combined with a metal activating agent, as in the composition according to the invention and this presents the disadvantage of not providing the surprising activity of the present invention and of lacking its universal character, i.e. not only in human beings but also in the whole animal and vegetable kingdoms.

The dispersing agent usable in the composition according to the invention may be liposomal, or micellar, or peptidic or in the form of microemulsion or of nanoemulsion, or in the form of microcapsule.

The activating agent may not be present in the composition according to the invention at doses exceeding ppm (or mg/L). It is only within these precise concentrations ranging from 0 to 20 ppm that the surprising effects described above and claimed in the present invention (i.e. a noticeable activation of the inhibiting potency of the active ingredient (or ingredients) present in the composition according to the invention) are observed.

In the case of a liposomal dispersing agent, this one may be advantageously chosen among the natural or synthetic liposomes of anionic or cationic type.

The first function of this liposomal dispersing agent is to lower the interfacial tension in order to emulsify the various active ingredients (or anti-infectious agents), which are themselves combined with metal activating agents.

The second function of this liposomal dispersing agent is to wrap up in its lipidic microsphere the various active ingredients which are combined with metal activating adjuvants.

In the case of a micellar dispersing agent, the active ingredient is not in a vesicle, but is inserted between two micelles.

In the case of a peptidic dispersing agent, this one constitutes a vector to which the active molecule is bound.

In the case of the microcapsule, the active ingredient may be coated with an alginate for example.

Activating Agent

The activating agent of the composition according to the invention consists of at least one metal element, which is alone or combined, but which never is in the form of a binding combination, i.e. which does not form a molecule with the active ingredient.

Thus, the activating agent may consist of only one unit metal element or a combination of several unit metal elements, which are not combined so as to form a new molecule, according to the above definition.

The metal element of the activating agent in the composition according to the invention may be constituted of all the known metals which are listed in Mendeleev's periodic table.

More particularly, the following elements are mentioned as metal element which may be advantageously included in the composition of the activating element: lithium, sodium, cadmium, cobalt, copper, zinc and tin, or others.

The activating agent of the composition according to the invention may come in different forms, for example in the form of a metal colloid which is nanoparticular or not, or a metal salt, or in the form of a metal ion, or in the form of a natural compound which already contains a group of metals as sodium-free and dehydrated sea water.

Among the activating agents usable within the scope of the present invention, more particularly and not restricted to these compounds, the fluorides or all other salt of lithium, sodium, tin, cadmium, cobalt, copper, zinc, etc. are mentioned.

Anti-Infectious Agent

The composition according to the invention may be normally applied to all the existing anti-infectious active ingredients (or agents) whatever their origin, natural or of chemical synthesis, and in particular to the following anti-infectious agents: all the bactericidal, antibiotic, fungicidal, virucidal, antiparasitic compounds, surface disinfectants, spermicides, phyto-sanitary compounds, whatever their chemical, vegetable or natural origin.

As antibiotics usable within the scope of the present invention, penicillins, cephalosporins, cyclines, aminosides, macrolides, sulfamides, quinolones, phenicolated antibiotics, etc. may be more especially mentioned.

As antibiotics usable within the scope of the present invention, lincosamides, synergistines, glycopeptidic antibiotics, fusidic acid, fosfomycin, rifampicin, etc. may also be mentioned.

As virucides usable within the scope of the present invention:
the chemical virucides, which may be chosen among quaternary ammonium compounds, for example benzalkonium chloride, and tetradecyl-dimethyl-benzyl-ammonium fluoride (or myristalkonium fluoride), etc. and
the virucides of vegetable origin, which may be chosen among the essential oils of oregano (Origanum majorana), thyme (Thymus vulgaris), and savory (Satureja montana), etc., or among plant extracts such as grapefruit (Citrus paradisi) seed extract, or
the virucides of natural origin chosen among the products of natural origin such as Propolis, etc. may be mentioned in particular.

As bactericides usable within the scope of the present invention, the chemical bactericides, such as quaternary ammonium compounds, biguanides, carbanilides, phenolic compounds, chlorinated compounds, and glutaraldehyde, etc. may be mentioned in particular.

According to a particularly advantageous mode of realization of the present invention, the anti-infectious agent is the following fluorinated quaternary ammonium compound: tetradecyl-dimethyl-benzyl-ammonium fluoride (or TDBAF or myristalkonium fluoride). With such an anti-infectious agent present in the composition according to the invention, this one may be administered by injection.

TDBAF belongs to the family of the cationic quaternary ammonium compounds which is known for its potent antibacterial properties, and benzalkonium chloride or alkyl-dimethyl-benzyl-ammonium chloride is one of its more active compounds. The activity of TDBAF was described in the following two publications: “Innocuity of tetradecyl-dimethyl-benzyl-ammonium fluoride on the DNA of human spermatozoa”, of Laforest G., Sergerie M., Bleau G, in Contraception, 2004 May; 69 (5): 425-32 (PMID: 15105067) [PubMed—indexed for Medline]; and “Efficacy of a new quaternary ammonium compound against TB”, of Byrne C., Healy T M, in Ir J Med Sci, 1999 January-March; 168 (1): 45-6 (PMID: 10098344) [PubMed—indexed for Medline]. [TB=Mycobacterium tuberculosis].

As fungicides usable within the scope of the present invention:
the chemical fungicides, for example econazole, ketoconazole, ciclopirox olamine, etc., and
the fungicides of vegetable origin, for example the essential oils of rose geranium (Pelargonium asperum), palmarosa (Cymbopogon martinii variety motia), and spike lavender (Lavandula latifolia), etc., or plant extracts such as grapefruit (Citrus paradisi) seed extract, or
the fungicides of natural origin such as Propolis, etc. may be mentioned in particular.

As spermicides usable within the scope of the present invention, the quaternary ammonium compounds (in particular benzalkonium chloride and tetradecyl-dimethyl-benzyl-ammonium fluoride), nonoxynol 9 and p-menthanylphenyl polyoxyethylene ether may be mentioned in particular.

As phyto-sanitary product usable within the scope of the present invention, Neem oil (extracted from Azadirachta indica) may be mentioned in particular.

The anti-infectious agent according to the invention may be of chemical origin (as it is the case for the antibiotics, the fungicides and the virucides), or of vegetable or natural origin.

Product of chemical origin means in the present invention all the products prepared by total or partial synthesis.

Product of vegetable origin means in the present invention the whole product of the plant or all type of plant extract, whatever the method of extraction or of production.

Product of natural origin means in the present invention a product the origin of which is neither chemical, nor vegetable, but animal, such as Propolis which is a natural product made by bees.

As products of vegetable origin usable within the scope of the present invention as anti-infectious agent, grapefruit seed extract, essential oils, macerated extracts, tinctures, dry extracts, aqueous extracts, and total extracts of plants, and all other type of plant extract, whatever the method of extraction or of production, are more particularly mentioned.

As product of natural origin usable within the scope of the present invention as anti-infectious agent, Propolis, which is a complex made by bees with their secretions and a series of resinous, gummy and balsamic substances is more particularly mentioned.

The composition according to the invention may be used in the fields of surface disinfection, hygiene products or cosmetic products, insofar as they contain an anti-infectious agent, as well as of phyto-sanitary products and food preservative agents.

Hence, the composition according to the invention may contain in addition one or several ingredients (or additional compounds) which are classically used in the concerned fields. The amounts of these various additional ingredients are those usually used in the concerned fields.

Of course, the specialist in the art will make sure to chose the possible compound or compounds to add to the composition according to the invention (in particular according to the envisaged utilization or application), as well as their concentration, so that the advantageous properties intrinsically linked should not be, or should not be substantially altered by the envisaged addition. In particular, the advantageous properties of the anti-infectious agent on the one hand, and of the dispersing agent and activating agent, on the other hand, will not have to be damaged by this (these) additional compound(s).

Thus, the object of the present invention is also to use the composition according to the invention in a cosmetic or body hygiene product.

The composition according to the invention may also be used as excipient in a phyto-sanitary product.

More generally, the composition according to the invention may be applied to the human or animal body as a whole, or to vegetable organisms, or to inert surfaces.

Finally, the object of the present invention is also the utilization, in a composition inhibiting or destroying at least one living or unicellular organism and containing at least one anti-infectious agent, of the combination of at least one activating agent containing at least one metal element in an amount not exceeding 20 mg/L in said composition with one dispersing agent which is liposomal, or micellar, or peptidic, or in the form of microemulsion or of nanoemulsion, or even in the form of microcapsule, in order to increase the inhibiting or destroying potency of the anti-infectious agent.

The utilization according to the invention of such a combination allows the activation of the activity of the anti-infectious agent, which in particular may be an antibiotic, bactericidal, fungicidal, or virucidal activity, which may relate to the whole human or animal body, or to surfaces to disinfect, or even to vegetable organisms. The importance of the activation may vary according to the microorganisms encountered but not the reality of the effect.

More particularly, the utilization according to the invention of such a combination allows the activation of a bactericidal activity to combat an infectious agent of the bacterial families. This effect may be applied to all bacterial families: gram-positive and gram-negative cocci, gram-positive and gram-negative bacilli, acid- and alcohol-fast bacilli, spiral bacteria, etc. This utilization for bactericidal purposes allows not only the inhibition of bacterial resistances (for example, in general, beta-lactamase-positive bacteria become beta-lactamase-negative) in increasing the efficacy of the active ingredients, but also allows the reduction of the doses of active ingredients, whatever the active ingredient, and thus it minimizes their possible adverse side effects in human beings, animals, plants and in the environment as a whole.

Furthermore, the utilization according to the invention of such a combination allows the activation of a fungicidal activity, which may be applied to the different forms of fungi: dermatophytes, yeasts, etc.

Finally, the utilization according to the invention of such a combination allows the activation of a spermicidal effect.

The invention is illustrated in greater detail in the following examples. In the examples, except where otherwise stated, all the amounts are expressed as a percentage in volume, or as mg/L, or as pg or as μL of composition.

### EXAMPLES

### Products

Microorganisms:
Staphylococcus aureus ATCC 29213,
MRSA (meticillin resistant Staphylococcus aureus),
Escherichia coli ATCC 25922,
Pseudomonas aeruginosa ATCC 27853, and
Streptococcus pneumoniae ATCC 49619.

Anti-infectious agents tested:
Thyme essential oil (Thymus vulgaris), called hereafter TEO,
Eucalyptus essential oil (Eucalyptus globulus), called hereafter EucEO,
Savory essential oil (Satureja montana), called hereafter SEO,
Clove bud essential oil (Eugenia caryophyllus),
Lavender essential oil (Lavandula officinalis),
Niaouli essential oil (Melaleuca quinquenervia),
Oregano essential oil (Origanum majorana),
Ravintsara essential oil (Cinnamomum camphora cineoliferum),
Rosemary essential oil (Rosmarinus officinalis),
Tea tree essential oil (Melaleuca alternifolia).
Grapefruit seed extract (Citrus paradisi), called hereafter GSE,

Dispersing agent: liposomal dispersing agent marketed by Sigene (Switzerland), under the commercial name of Disper®; it is a natural emulsifying complex consisting in particular of an alcoholic extract of vegetable cell membranes containing alcohol, water, sweet almond (Prunus dulcis) extract, lecithin, oleic acid, vitamine C and vitamine E. This dispersing agent was used in the above-mentioned examples but it is not specific and may be replaced by other liposomal, or micellar, etc. agents.

Metal activating agents:
Lithium fluoride (LiF),
Cobalt fluoride (CoF₂),
Stannous fluoride (SnF₂).

### Material and Method

First, the aim is to determine the percentage of inhibition of two bacterial strains, each strain being inserted in an inoculum, by an active ingredient (or anti-infectious agent), in the presence of a composition activating the inhibiting potency, comprising at least one metal activating agent combined with (according to the present invention) or not combined with (according to the prior art) a dispersing agent.

With regard to the present invention, the percentage of inhibition means the percentage (in % vol/vol) of active ingredient necessary to inhibit 100 μL of inoculum.

Secondly, in order to state the results with more precision, the aim is to determine the minimal inhibitory concentration (MIC) of an active ingredient (or anti-infectious agent) against five bacterial strains, each strain being inserted in an inoculum, in the presence of a composition activating the inhibiting potency, comprising at least one metal activating agent combined with a dispersing agent, according to the present invention.

With regard to the present invention, the minimal inhibitory concentration (MIC) means the minimal concentration (in % vol/vol and μL) of active ingredient necessary to inhibit 100 μL of inoculum.

To determine the percentage of inhibition, the following compositions are prepared for each example:
a sample of positive control (called letter B in the test tables), which only contains the inoculum (100 μL) in the sterile culture medium of the microorganisms (100 μL).
a sample of negative control (called letter M in the test tables), which only contains the sterile culture medium of the microorganisms (200 μL),
a first comparative inhibiting composition CCi, containing 100 μL of active ingredient (i corresponding to a whole number), to which 100 μL of inoculum are added,
a second comparative inhibiting composition CCᵢ₊₁, containing 50 μL of active ingredient and 50 μL of metal activating agent (total volume of 100 μL), to which 100 μL of inoculum are added,
a third comparative inhibiting composition CC₁₊₂, containing 33.3 μL of active ingredient and 66.6 μL of a mixture of two metal activating agents in equal parts (total volume of 100 μL), to which 100 μL of inoculum are added, and
one or several inhibiting compositions according to the present invention, Ci (i corresponding to a whole number), containing 100 μL of a mixture of active ingredient, metal activator and dispersing agent, keeping to a total volume of 100 μL, to which 100 μL of inoculum are added.

To determine the MIC, the following compositions are prepared for each example:
a sample of positive control (called letter B in the test tables), which only contains the inoculum (100 μL) in the sterile culture medium of the microorganisms (100 μL).
a sample of negative control (called letter M in the test tables), which only contains the sterile culture medium of the microorganisms (200 μL), and
one or several inhibiting compositions according to the present invention, Ci (i corresponding to a whole number), containing 100 μL of a mixture of active ingredient, metal activator and dispersing agent, to which 100 μL of inoculum are added.

The trials were carried out with the five above-mentioned bacterial strains according to the method of microdilution in liquid medium described by the CLSI (Clinical and Laboratory Standards Institute), which was adapted if necessary. The method may be summarized as follows:
distribute 100 μL of sterile culture medium in the pits of positive control of the 96-well, U-bottom plate,
Add to each appropriate pit of the 96-well plate:

1) 100 μL of an anti-infectious agent, or

100 μL of a mixture of an anti-infectious agent and one or several activating agents in equal parts, or

10 μL of an anti-infectious agent and 90 μL of dispersing agent, or

10 μL of a mixture of an anti-infectious agent and one or several activating agents in equal parts and 90 μL of dispersing agent,

keeping to a total volume of 100 μL, except in the pits of positive control and negative control.

2) 100 μL of inoculum with an end concentration of 3-5×10 ⁵ CFU/mL (colony-forming units per milliliter), except in the pits of negative control.
Distribute 200 μL of sterile culture medium in the pits of negative control of the 96-well plate.
The plate is covered and incubated at 37° C. for 18 to 24 hours.
The results are read with a reading-mirror or a spectrophotometer at the optical density of 620 nm.

The sensitivity threshold is not available in the CLSI (Clinical and Laboratory Standards Institute) guide. These trials were carried out in the Laboratory of medical microbiology of the university hospital Center of Montreal of the Hôtel Dieu Hospital, Research and Development Unit, Montreal, Canada.

### Example 1

### Activation of the Inhibiting Activity of Thyme Essential Oil (Thymus vulgaris) (TEO)

In addition to the compositions of control described above (B positive control and M negative control), the following compositions were prepared in order to show the action of metal activating agent(s) alone or combined with a liposomal dispersing agent:
a first comparative inhibiting composition CC₁, containing 100 μL of TEO,
a second comparative inhibiting composition CC₂, containing 50 μL of TEO and 50 μL of LiF, i.e. a total volume of 100 μL,
a third comparative inhibiting composition CC₃, containing 33.3 μL of TEO and 33.3 μL of LiF+33.3 μL of SnF₂, i.e. a total volume of 100 L,
a fourth comparative inhibiting composition CC₄, containing, for a total volume of 100 μL:
   10 μL of TEO, and
   90 μL of Disper®,
a first inhibiting composition C₁ according to the invention, containing, for a total volume of 100 μL:
   10 μL of the following mixture: TEO (5 μL)+LiF (5 μL), and
   90 μL of Disper®,
a second inhibiting composition C₂ according to the invention, containing, for a total volume of 100 μL:
   10 μL of the following mixture: TEO (3.33 μL)+LiF (3.33 μL)+SnF₂ (3.33 μL), and
   90 μL of Disper®.

100 μL of inoculum are added to each of the compositions described above.

The end concentration of LiF in the medium, i.e. in the 200 μL of each appropriate pit, is 8 mg/L. Therefore there are 1.6 μg of LiF in these 200 μL. The end concentration of SnF₂ in this medium is 5 mg/L, i.e. there is 1 μg of SnF₂ in the 200 μL of each appropriate pit.

For each of these compositions, the percentage of inhibition of 2 bacterial strains by the active ingredient (thyme essential oil in this example) was determined. The experimental results are presented in table 1 below.

TABLE 1

| | | | Percentage (% v/v) of inhibition of the bacterial | | |
|---|---|---|---|---|---|
| | | | strains by TEO alone or combined with LiF (8 mg/L)¹ | | |
| | | | or with LiF (8 mg/L)¹ + SnF₂ (5 mg/L)¹, | | |
| | | | with or without Disper ®² | | |
| | | | | | CC₃ |
| | | | | | TEO |
| | | | | CC₂ | (33.3 μL = 16.6%)³ + |
| | | | | TEO | LiF |
| Bacterial | Negative | Positive | CC₁ | (50 μL = 25%)³ + | (33.3 μL = 16.6%)³ + |
| strains | control | control | TEO | LiF | SnF₂ |
| (100 μL of inoculum) | M⁴ | B⁵ | (100 μL = 50%)³ | (50 μL = 25%)³ | (33.3 μL = 16.6%)³ |
| Staphylococcus aureus | − | + | 79 | 93 | 92 |
| ATCC 29213 | | | | | |
| MRSA | − | + | 79 | 93 | 93 |
| | | | Percentage (% v/v) of inhibition of the bacterial | | |
| | | | strains by TEO alone or combined with LiF (8 mg/L)¹ | | |
| | | | or with LiF (8 mg/L)¹ + SnF₂ (5 mg/L)¹, | | |
| | | | with or without Disper ®² | | |
| | | | | | C₂ |
| | | | | | TEO |
| | | | | C₁ | (3.33 μL = 1.66%)³ + |
| | | | | TEO | LiF |
| | | | CC₄ | (5 μL = 2.5%)³ + | (3.33 μL = 1.66%)³ + |
| | | | TEO | LiF | SnF₂ |
| Bacterial | Negative | Positive | (10 μL = 5%)³ + | (5 μL = 2.5%)³ + | (3.33 μL = 1.66%)³ + |
| strains | control | control | Disper ® | Disper ® | Disper ® |
| (100 μL of inoculum) | M⁴ | B⁵ | (90 μL = 45%)³ | (90 μL = 45%)³ | (90 μL = 45%)³ |
| Staphylococcusaureus | − | + | 85 | 89 | 87 |
| ATCC 29213 | | | | | |
| MRSA | − | + | 81 | 87 | 85 |

¹End concentrations of LiF and SnF₂ in the medium, i.e. 1.6 μg of LiF ± 1 μg of SnF₂ in the 200 μL of each appropriate pit.
²[(TEO) or (TEO + LiF) or (TEO + LiF + SnF₂)] + [Disper] [10:90].
³Percentage of each product in the 200 μL of each appropriate pit.
⁴Negative control (sterile culture medium): 0% bacterial growth.
⁵Positive control (inoculum): 100% bacterial growth.

The experimental results presented in table 1 show that the inhibiting compositions according to the invention allow the inhibition of bacteria with doses of active ingredients much lower (1.66% or 2.5% in example 1) than those necessary when the active ingredients are used alone (50% in this example 1) or when the active ingredients are only combined with metal activating agents (without dispersing agent, of liposomal type for example), as in the prior art (25% in this example 1).

The composition C₂ according to the invention contains 30 times less thyme essential oil than the comparative composition CC₁, containing the same active ingredient without either activator or dispersing agent, and it has a greater inhibiting potency (85% or 87% instead of 79%).

For the microorganism Staphylococcus aureus ATCC 29213, the addition of LiF to thyme essential oil (composition CC₂) allowed an increase in the inhibiting potency of thyme essential oil from 79% to 93%, while reducing by half the percentage of thyme essential oil used which goes down from 50% to 25%. The addition of SnF₂ to LiF (composition CC₃) does not directly increases the inhibiting value. But, for a not very different result (92% of inhibition instead of 93%), it allows the reduction of the percentage of thyme essential oil used to 16.6%. The combination of the dispersing agent with LiF in the composition C₁ according to the invention increases the efficacy of thyme essential oil (89% instead of 79%) while significantly reducing the percentage of thyme essential oil used, which goes down from 50% to 2.5%. In the composition C₂ according to the invention, the double metal activation of LiF and SnF₂ allows, added to the dispersing agent, the reduction of the amount of thyme essential oil to 1.66%. Therefore, the composition C₂ according to the invention allows the division by 30 of the amount of thyme essential oil sufficient to obtain an inhibiting potency superior to that observed with thyme essential oil alone, i.e. 87% instead of 79%.

For the microorganism meticillin resistant Staphylococcus aureus (MRSA), the results are similar to those obtained with the reference strain of Staphylococcus aureus ATCC 29213. The combination of the dispersing agent with LiF in the composition C₁ according to the invention increases the efficacy of thyme essential oil (87% instead of 79%), while allowing the reduction of the percentage of thyme essential oil used: 2.5% instead of 50%. In the composition C₂ according to the invention, the double metal activation of LiF and SnF₂ allows, added to the dispersing agent, the reduction of the amount of thyme essential oil to 1.66%. Therefore, the composition C₂ according to the invention allows the division by 30 of the amount of thyme essential oil sufficient to obtain an inhibiting potency superior to that observed with thyme essential oil alone, i.e. 85% instead of 79%.

These experimental results show that the inhibiting compositions according to the invention allow the reduction of the doses of active ingredients used, while increasing their inhibiting potency.

### Example 2

### Activation of the Inhibiting Activity of Eucalyptus Essential Oil (Eucalyptus globulus) (EucEO)

In addition to the compositions of control described above (B positive control and M negative control), the following compositions were prepared in order to show the action of metal activating agent(s) alone or combined with a liposomal dispersing agent:
a first comparative inhibiting composition CC₅, containing 100 μL of EucEO,
a second comparative inhibiting composition CC₆, containing 50 μL of EucEO and 50 μL of LiF, i.e. a total volume of 100 μL,
a third comparative inhibiting composition CC₇, containing 33.3 μL of EucEO and 33.3 μL of LiF+33.3 μL of SnF₂, i.e. a total volume of 100 μL,
a fourth comparative inhibiting composition CC₈, containing, for a total volume of 100 μL:
   10 μL of EucEO, and
   90 μL of Disper®,
a first inhibiting composition C₃ according to the invention, containing, for a total volume of 100 μL:
   10 μL of the following mixture: EucEO (5 μL)+LiF (5 μL), and
   90 μL of Disper®,
a second inhibiting composition C₄ according to the invention, containing, for a total volume of 100 μL:
   10 μL of the following mixture: EucEO (3.33 μL)+LiF (3.33 μL)+SnF₂ (3.33 μL), and
   90 μL of Disper®.

100 μL of inoculum are added to each of the compositions described above.

The end concentration of LiF in the medium, i.e. in the 200 μL of each appropriate pit, is 8 mg/L. Therefore there are 1.6 pg of LiF in these 200 μL. The end concentration of SnF₂ in this medium is 5 mg/L, i.e. there is 1 μg of SnF₂ in the 200 μL of each appropriate pit.

For each of these compositions, the percentage of inhibition of 2 bacterial strains by the active ingredient (eucalyptus essential oil in this example) was determined. The experimental results are presented in table 2 below.

TABLE 2

| | | | Percentage (% v/v) of inhibition of the | | |
|---|---|---|---|---|---|
| | | | bacterial strains by EucEO alone or combined | | |
| | | | with LiF (8 mg/L)¹ | | |
| | | | or with LiF (8 mg/L)¹ + SnF₂ (5 mg/L)¹, | | |
| | | | with or without Disper ®² | | |
| | | | | CC₆ | CC₇ |
| | | | | EucEO | EucEO |
| | | | | (50 μL = | (33.3 μL = 16.6%)³ + |
| Bacterial | | | | 25%)³ + | LiF |
| strains | Negative | Positive | CC₅ | LiF | (33.3 μL = 16.6%)³ + |
| (100 μL | control | control | EucEO | (50 μL = | SnF₂ |
| of inoculum) | M⁴ | B⁵ | (100 μL = 50%)³ | 25%)³ | (33.3 μL = 16.6%)³ |
| Staphylococcus | − | + | 80 | 95 | 92 |
| aureus | | | | | |
| ATCC 29213 | | | | | |
| MRSA | − | + | 80 | 92 | 90 |
| | | | Percentage (% v/v) of inhibition of the | | |
| | | | bacterial strains by EucEO alone or combined | | |
| | | | with LiF (8 mg/L)¹ | | |
| | | | or with LiF (8 mg/L)¹ + SnF₂ (5 mg/L)¹, with | | |
| | | | or without Disper ®² | | |
| | | | | | C₄ |
| | | | | | EucEO |
| | | | | C₃ | (3.33 μL = 1.66%)³ + |
| | | | CC₈ | EucEO | LiF |
| | | | EucEO | (5 μL = 2.5%)³ + | (3.33 μL = 1.66%)³ + |
| Bacterial | | | (10 μL = 5%)³ + | LiF | SnF₂ |
| strains | Negative | Positive | Disper ® | (5 μL = 2.5%)³ + | (3.33 μL = 1.66%)³ + |
| (100 μL | control | control | (90 μL = | Disper ® | Disper ® |
| of inoculum) | M⁴ | B⁵ | 45%)³ | (90 μL = 45%)³ | (90 μL = 45%)³ |
| Staphylococcus | − | + | 84 | 94 | 90 |
| aureus | | | | | |
| ATCC 29213 | | | | | |
| MRSA | − | + | 82 | 91 | 86 |

¹End concentrations of LiF and SnF₂ in the medium, i.e. 1.6 μg of LiF ± 1 μg of SnF₂ in the 200 μL of each appropriate pit.
²[(EucEO) or (EucEO + LiF) or (EucEO + LiF + SnF₂)] + [Disper] [10:90].
³Percentage of each product in the 200 μL of each appropriate pit.
⁴Negative control (sterile culture medium): 0% bacterial growth.
⁵Positive control (inoculum): 100% bacterial growth.

In this trial with eucalyptus essential oil, results similar to those obtained in example 1 with thyme essential oil are observed.

The experimental results presented in table 2 show that the inhibiting compositions according to the invention allow the inhibition of bacteria with doses of active ingredients much lower (1.66% or 2.5% in example 2) than those necessary when the active ingredients are used alone (50% in this example 2) or when the active ingredients are only combined with metal activating agents (without dispersing agent, of liposomal type for example), as in the prior art (25% in this example 2).

The composition C₄ according to the invention contains 30 times less eucalyptus essential oil than the comparative composition CC_{b}, containing the same active ingredient without either activator or dispersing agent, and it has a greater inhibiting potency (86% or 90% instead of 80%).

For the microorganism Staphylococcus aureus ATCC 29213, the addition of LiF to eucalyptus essential oil (composition CC₆) allowed an increase in the inhibiting potency of eucalyptus essential oil from 80% to 95%, while reducing by half the percentage of eucalyptus essential oil used which goes down from 50% to 25%. The addition of SnF₂ to LiF (composition CC₇) does not directly increase the inhibiting value. But, for a not very different result (92% of inhibition instead of 95%), it allows the reduction of the percentage of eucalyptus essential oil used to 16.6%. The combination of the dispersing agent with LiF in the composition C₃ according to the invention increases the efficacy of eucalyptus essential oil (94% instead of 80%) while significantly reducing the percentage of eucalyptus essential oil used, which goes down from 50% to 2.5%.

In the composition C₄ according to the invention, the double metal activation of LiF and SnF₂ allows, added to the dispersing agent, the reduction of the amount of eucalyptus essential oil to 1.66%. Therefore, the composition C₄ according to the invention allows the division by 30 of the amount of eucalyptus essential oil sufficient to obtain an inhibiting potency superior to that observed with eucalyptus essential oil alone, i.e. 90% instead of 80%.

For the microorganism meticillin resistant Staphylococcus aureus (MRSA), the results are similar to those obtained with the reference strain of Staphylococcus aureus ATCC 29213, as in example 1 with thyme essential oil. The combination of the dispersing agent with LiF in the composition C₃ according to the invention increases the efficacy of eucalyptus essential oil (91% instead of 80%), while allowing the reduction of the percentage of eucalyptus essential oil used: 2.5% instead of 50%. In the composition C₄ according to the invention, the double metal activation of LiF and SnF₂ allows, added to the dispersing agent, the reduction of the amount of eucalyptus essential oil to 1.66%.

Therefore, the composition C₄ according to the invention allows the division by 30 of the amount of eucalyptus essential oil sufficient to obtain an inhibiting potency superior to that observed with eucalyptus essential oil alone, i.e. 86% instead of 80%.

These experimental results show that the inhibiting compositions according to the invention allow the reduction of the doses of active ingredients used, while increasing their inhibiting potency.

### Example 3

### Activation of the Inhibiting Activity of Savory Essential Oil (Satureja montana) (SEO)

In addition to the compositions of control described above (B positive control and M negative control), the following compositions were prepared in order to show the action of metal activating agent(s) alone or combined with a liposomal dispersing agent:
a first inhibiting composition C₅ according to the invention, containing, for a total volume of 100 μL:
   10 μL of the following mixture: SEO (3.33 μL)+LiF (3.33 μL)+SnF₂ (3.33 μL), and
   90 μL of Disper®,
a second inhibiting composition C₆ according to the invention, containing, for a total volume of 100 μL:
   10 μL of the following mixture: SEO (3.33 μL)+LiF (3.33 μL)+CoF₂ (3.33 μL), and
   90 μL of Disper®.

100 μL of inoculum are added to each of the compositions described above.

The end concentration of LiF in the medium, i.e. in the 200 μL of each appropriate pit, is 8 mg/L. Therefore there are 1.6 μg of LiF in these 200 μL. The end concentrations of SnF₂ and Co F₂ in this medium are 5 mg/L and 4 mg/L respectively, i.e. there are 1 μg of SnF₂ or 0.8 μg de CoF₂ in the 200 μL of each appropriate pit.

For each of these compositions, the MIC of the active ingredient (savory essential oil in this example) was determined on 5 microorganisms. The experimental results of these trials are presented in table 3 below.

TABLE 3

| | | | MIC (% v/v) of the bacterial strains by SEO | |
|---|---|---|---|---|
| | | | combined with LiF (8 mg/L)¹ + SnF₂ (5 mg/L)¹, or | |
| | | | with LiF (8 mg/L)¹ + CoF₂ (4 mg/L)¹, with Disper ®² | |
| | | | C₅ | C₆ |
| Bacterial | | | SEO (3.33 μL = 1.66%)³ + | SEO (3.33 μL = 1.66%)³ + |
| strains | Negative | Positive | LiF (3.33 μL = 1.66%)³ + | LiF (3.33 μL = 1.66%)³ + |
| (100 μL | control | control | SnF₂ (3.33 μL = 1.66%)³ + | CoF₂ (3.33 μL = 1.66%)³ + |
| of inoculum) | M⁴ | B⁵ | Disper ® (90 μL = 45%)³ | Disper ® (90 μL = 45%)³ |
| Staphylococcus | − | + | 0.025 | 0.05 |
| aureus ATCC 29213 | | | | |
| MRSA | − | + | 0.05 | 0.1 |
| Pseudomonas aeruginosa ATCC 27853 | − | + | 0.05 | 0.1 |
| Escherichia coli ATCC 25922 | − | + | 0.1 | 0.1 |
| Streptococcus pneumoniae ATCC 49619 | − | + | 0.1 | 0.1 |

¹End concentrations of LiF, SnF₂ and CoF₂ in the medium, i.e. 1.6 μg of LiF + 1 μg of SnF₂ or 1.6 μg of LiF + 0.8 μg of CoF₂ in the 200 μL of each appropriate pit.
²[(SEO + LiF + SnF₂) or (SEO + LiF + CoF₂)] + [Disper] [10:90].
³Percentage of each product in the 200 μL of each appropriate pit.
⁴Negative control (sterile culture medium): 0% bacterial growth.
⁵Positive control (inoculum): 100% bacterial growth.

The experimental results presented in table 3 show that the inhibiting compositions according to the invention allow the inhibition of bacteria with doses of active ingredients much lower than those necessary when the inhibiting compositions only contain metal activating agents (without dispersing agent, of liposomal type for example). In the compositions C₅ and C₆ according to the invention, the double metal activation of LiF and SnF₂ and that of LiF and Co F₂ allow, added to the dispersing agent, the reduction of the amount of savory essential oil to 1.66%, as in the examples 1 and 2 for the other essential oils tested.

For the microorganisms Streptococcus pneumoniae ATCC 49619 and Escherichia coli ATCC 25922, the effect of both compositions according to the invention C₅ and C₆ is equivalent in terms of MIC.

On the other hand, regarding the microorganisms Staphylococcus aureus ATCC 29213, MRSA (meticillin resistant Staphylococcus aureus), and Pseudomonas aeruginosa ATCC 27853, it should be noted that stannous fluoride has a greater catalytic effect than that of cobalt fluoride when each fluoride is combined with lithium fluoride and Disper®. This improved catalytic effect results in a lower MIC with the composition C₅ based on stannous fluoride.

### Example 4

### Activation of the Inhibiting Activity of Thyme Essential Oil (Thymus vulgaris) (TEO)

In addition to the compositions of control described above (B positive control and M negative control), the following compositions were prepared in order to show the action of metal activating agent(s) alone or combined with a liposomal dispersing agent:
a first inhibiting composition C₇ according to the invention, containing, for a total volume of 100 μL:
   10 μL of the following mixture: TEO (3.33 μL)+LiF (3.33 μL)+SnF₂ (3.33 μL), and
   90 μL of Disper®,
a second inhibiting composition C₈ according to the invention, containing, for a total volume of 100 μL:
   10 μL of the following mixture: TEO (3.33 μL)+LiF (3.33 μL)+CoF₂ (3.33 μL), and
   90 μL of Disper®.

100 μL of inoculum are added to each of the compositions described above.

The end concentration of LiF in the medium, i.e. in the 200 μL of each appropriate pit, is 8 mg/L. Therefore there are 1.6 μg of LiF in these 200 μL. The end concentrations of SnF₂ and CoF₂ in this medium are 5 mg/L and 4 mg/L respectively, i.e. there are 1 μg of SnF₂ or 0.8 μg de CoF₂ in the 200 μL of each appropriate pit.

For each of these compositions, the MIC of the active ingredient is determined on 5 microorganisms. The experimental results of these trials are presented in table 4 below.

TABLE 4

| | | | MIC (% v/v) of the bacterial strains by TEO combined | |
|---|---|---|---|---|
| | | | with LiF (8 mg/L)¹ + SnF₂ (5 mg/L)¹, or with LiF | |
| | | | (8 mg/L)¹ + CoF₂ (4 mg/L)¹, with Disper ®² | |
| | | | C₇ | C₈ |
| Bacterial | | | TEO (3.33 μL = 1.66%)³ + | TEO (3.33 μL = 1.66%)³ + |
| strains | Negative | Positive | LiF (3.33 μL = 1.66%)³ + | LiF (3.33 μL = 1.66%)³ + |
| (100 μL | control | control | SnF₂ (3.33 μL = 1.66%)³ + | CoF₂ (3.33 μL = 1.66%)³ + |
| of inoculum) | M⁴ | B⁵ | Disper ® (90 μL = 45%)³ | Disper ® (90 μL = 45%)³ |
| Staphylococcus | − | + | 0.05 | 0.1 |
| aureus ATCC 29213 | | | | |
| MRSA | − | + | 0.2 | 0.1 |
| Pseudomonas aeruginosa ATCC 27853 | − | + | 0.05 | 0.1 |
| Escherichia coli ATCC 25922 | − | + | 0.05 | 0.05 |
| Streptococcus pneumoniae ATCC 49619 | − | + | 0.1 | 0.05 |

¹End concentrations of LiF, SnF₂ and CoF₂ in the medium, i.e. 1.6 μg of LiF + 1 μg of SnF₂ or 1.6 μg of LiF + 0.8 μg of CoF₂ in the 200 μL of each appropriate pit.
²[(TEO + LiF + SnF₂) or (TEO + LiF + CoF₂)] + [Disper] 10:90].
³Percentage of each product in the 200 μL of each appropriate pit.
⁴Negative control (sterile culture medium): 0% bacterial growth.
⁵Positive control (inoculum): 100% bacterial growth.

As in the examples 1 to 3, the experimental results presented in table 4 show that the inhibiting compositions according to the invention allow the inhibition of bacteria with doses of active ingredients much lower than those necessary when the inhibiting compositions only contain metal activating agents (without dispersing agent, of liposomal type for example).

In the compositions C₇ et C₈ according to the invention, the double metal activation of LiF and SnF₂ and that of LiF and CoF₂ allow, added to the dispersing agent, the reduction of the amount of thyme essential oil to 1.66%, as in the above examples for the other essential oils tested.

For the microorganism Escherichia coli ATCC 25922, the effect of both compositions according to the invention C₇ and C₈ is equivalent in terms of MIC.

For the microorganisms MRSA (meticillin resistant Staphylococcus aureus), and Streptococcus pneumoniae ATCC 49619, it should be noted that cobalt fluoride has a greater catalytic effect than that of stannous fluoride when they are combined with lithium fluoride and Disper®. This improved catalytic effect results in a lower MIC with the composition C₈ based on cobalt fluoride.

On the other hand, regarding the microorganisms Staphylococcus aureus ATCC 29213, and Pseudomonas aeruginosa ATCC 27853, we note that stannous fluoride has a greater catalytic effect than that of cobalt fluoride when they are combined with lithium fluoride and Disper®, and it results in a lower MIC with the composition C₇ based on stannous fluoride.

### Examples 5 to 12

### Activation of the Inhibiting Activity of the Essential Oils of eucalyptus (Eucalyptus globulus), clove Bud (Eugenia caryophyllus), lavender (Lavandula officinalis), niaouli (Melaleuca quinquenervia), oregano (Origanum majorana), ravintsara (Cinnamomum camphora cineoliferum), rosemary (Rosmarinus officinalis) and tea tree (Melaleuca alternifolia)

As in the examples 3 and 4, the following inhibiting compositions were tested:
a first group of inhibiting compositions C₉, C₁₁, C₁₃, C₁₅, C₁₇, C₁₉, C₂₁ and C₂₃ according to the invention, containing, for a total volume of 100 μL:
   10 μL of the following mixture: essential oil (3.33 μL)+LiF (3.33 μL)+SnF₂ (3.33 μL), and
   90 μL of Disper®,
a second group of inhibiting compositions C₁₀, C₁₂, C₁₄, C₁₆, C₁₈, C₂₀, C₂₂ and C₂₄ according to the invention, containing, for a total volume of 100 μL:
   10 μL of the following mixture: essential oil (3.33 μL)+LiF (3.33 μL)+CoF₂ (3.33 μL), and
   90 μL of Disper®.

100 μL of inoculum are added to each of the compositions described above.

The end concentration of LiF in the medium, i.e. in the 200 μL of each appropriate pit, is 8 mg/L. Therefore there are 1.6 μg of LiF in these 200 μL. The end concentrations of SnF₂ and CoF₂ in this medium are 5 mg/L and 4 mg/L respectively, i.e. there are 1 μg of SnF₂ or 0.8 μg de CoF₂ in the 200 μL of each appropriate pit.

For each of these compositions, the MIC of the active ingredient is determined on 5 microorganisms. The experimental results of these trials are presented in table 5 below.

TABLE 5
MIC (% v/v) of the bacterial strains by the essential oils (EO) combined with
LiF (8 mg/L)¹ + SnF₂ (5 mg/L)¹, or with LiF (8 mg/L)¹ + CoF₂ (4 mg/L)¹, with Disper ®²

| | Bacterial strains | | | | |
|---|---|---|---|---|---|
| | Staphylococcus | | Pseudomonas | Escherichia | Streptococcus |
| Test | aureus | | aeruginosa | coli | pneumoniae |
| products | ATCC 29213 | MRSA | ATCC 27853 | ATCC 25922 | ATCC 49619 |

Essential oil of OREGANO (Origanum majorana)

| | | | | | |
|---|---|---|---|---|---|
| C₉: LiF + SnF₂ + | 0.2 | 0.025 | 0.4 | 0.8 | 0.2 |
| Disper | | | | | |
| C₁₀: LiF + CoF₂ + | 0.4 | 0.1 | 0.4 | 0.8 | 0.1 |
| Disper | | | | | |

Essential oil of CLOVE BUD (Eugenia caryophyllus)

| | | | | | |
|---|---|---|---|---|---|
| C₁₁: LiF + SnF₂ + | 0.8 | 0.2 | 0.4 | 0.4 | 0.05 |
| Disper | | | | | |
| C₁₂: LiF + CoF₂ + | 0.8 | 0.2 | 0.4 | 0.4 | 0.05 |
| Disper | | | | | |

Essential oil of ROSEMARY (Rosmarinus officinalis)

| | | | | | |
|---|---|---|---|---|---|
| C₁₃: LiF + SnF₂ + | 0.2 | 0.1 | 0.8 | 0.8 | 0.2 |
| Disper | | | | | |
| C₁₄: LiF + CoF₂ + | 0.2 | 0.05 | 1.6 | 0.8 | 0.2 |
| Disper | | | | | |

Essential oil of TEA TREE (Melaleuca alternifolia)

| | | | | | |
|---|---|---|---|---|---|
| C₁₅: LiF + SnF₂ + | 0.1 | 0.05 | 1.6 | 0.8 | 0.4 |
| Disper | | | | | |
| C₁₆: LiF + CoF₂ + | 0.2 | 0.1 | 0.8 | 0.8 | 0.4 |
| Disper | | | | | |

Essential oil of RAVINTSARA (Cinnamomum camphora cineoliferum)

| | | | | | |
|---|---|---|---|---|---|
| C₁₇: LiF + SnF₂ + | 0.4 | 0.05 | 1.6 | 0.8 | 0.2 |
| Disper | | | | | |
| C₁₈: LiF + CoF₂ + | 0.2 | 0.05 | 1.6 | 0.8 | 0.2 |
| Disper | | | | | |

Essential oil of LAVENDER (Lavandula officinalis)

| | | | | | |
|---|---|---|---|---|---|
| C₁₉: LiF + SnF₂ + | 0.8 | 0.4 | >1.6 | 0.4 | 0.2 |
| Disper | | | | | |
| C₂₀: LiF + CoF₂ + | 0.8 | 0.2 | >1.6 | 0.4 | 0.4 |
| Disper | | | | | |

Essential oil of NIAOULI (Melaleuca quinquenervia)

| | | | | | |
|---|---|---|---|---|---|
| C₂₁: LiF + SnF₂ + | 0.2 | <0.012 | 1.6 | 1.6 | 0.2 |
| Disper | | | | | |
| C₂₂: LiF + CoF₂ + | 0.4 | <0.012 | 1.6 | 1.6 | 0.2 |
| Disper | | | | | |

Essential oil of EUCALYPTUS (Eucalyptus globulus)

| | | | | | |
|---|---|---|---|---|---|
| C₂₃: LiF + SnF₂ + | 0.8 | 0.2 | 1.6 | 1.6 | 0.2 |
| Disper | | | | | |
| C₂₄: LiF + CoF₂ + | 0.8 | 0.1 | 1.6 | 1.6 | 0.4 |
| Disper | | | | | |

¹End concentrations of LiF, SnF₂ and CoF₂ in the medium, i.e. 1.6 μg of LiF + 1 μg of SnF₂ or 1.6 μg of LiF + 0.8 μg of CoF₂ in the 200 μL of each appropriate pit.
²[(EO + LiF + SnF₂) or (EO + LiF + CoF₂)] + [Disper] [10:90].

The percentage of each product in the culture medium is the same one as that in tables 3 and 4: 1.66% of each essential oil (3.33 μL), 1.66% of LiF (3.33 μL)+1.66% of SnF₂ or of CoF₂ (3.33 μL) and 45% of Disper® (90 μL).

As in the examples 1 to 4, the experimental results presented in table 5 show that the inhibiting compositions according to the invention allow the inhibition of bacteria with doses of active ingredients much lower than those necessary when the inhibiting compositions only contain metal activating agents (without dispersing agent, of liposomal type for example).

According to the microorganisms and essential oils, stannous fluoride has a catalytic effect greater than, equivalent to or lower than that of cobalt fluoride, when it is combined with lithium fluoride and Disper®, with the essential oils.

For the microorganism Escherichia coli ATCC 25922, for example, the effect of both compositions according to the invention Cᵢ et Cᵢ₊₁ is equivalent in terms of MIC for all the essential oils tested.

For both groups of compositions C₉ to C₂₄ according to the invention, the double metal activation of lithium fluoride and stannous fluoride and that of lithium fluoride and cobalt fluoride allow, added to the dispersing agent, the reduction of the amount of the essential oils to 1.66%, as in the above examples, for all the microorganisms tested.

### Example 13

### Activation of the Inhibiting Activity of Grapefuit Seed Extract (Citrus paradisi) (GSE)

In addition to the compositions of control described above (B positive control and M negative control), the following compositions were prepared in order to show the action of metal activating agent(s) alone or combined with a liposomal dispersing agent:
a first comparative inhibiting composition CC₉, containing 100 μL of GSE,
a second comparative inhibiting composition CC₁₀, containing 50 μL of GSE and 50 μL of LiF, i.e. a total volume of 100 μL,
a first inhibiting composition C₂₅ according to the invention, containing, for a total volume of 100 μL:
   10 μL of the following mixture: GSE (5 μL)+LiF (5 μL), and
   90 μL of Disper®,
a second inhibiting composition C₂₆ according to the invention, containing, for a total volume of 100 μL:
   10 μL of the following mixture: GSE (3.33 μL)+LiF (3.33 μL) +SnF₂ (3.33 μL), and
   90 μL of Disper®,
a third inhibiting composition C₂₇ according to the invention, containing, for a total volume of 100 μL:
   10 μL of the following mixture: GSE (3.33 μL)+LiF (3.33 μL)+CoF₂ (3.33 μL), and
   90 μL of Disper®.

100 μL of inoculum are added to each of the compositions described above.

The end concentration of LiF in the medium, i.e. in the 200 μL of each appropriate pit, is 8 mg/L. Therefore there are 1.6 μg of LiF in these 200 μL. The end concentrations of SnF₂ and CoF₂ in this medium are 5 mg/L and 4 mg/L respectively, i.e. there are 1 μg of SnF₂ or 0.8 μg de CoF₂ in the 200 μL of each appropriate pit.

For each of the compositions C₂₅ to C₂₇, the MIC of the active ingredient is determined on 5 microorganisms. The experimental results of these trials are presented in table 6 below.

TABLE 6

| | | | MIC (% v/v and μL¹) of the bacterial strains | |
|---|---|---|---|---|
| | | | by GSE alone or combined with | |
| | | | LiF (8 mg/L)², or with LiF (8 mg/L)² + Disper ®³, or | |
| | | | with LiF (8 mg/L)² + SnF₂ (5 mg/L)² + Disper ®³, or | |
| Bacterial | | | with LiF (8 mg/L)² + CoF₂ (4 mg/L)² + Disper ®³ | |
| strains | Negative | Positive | CC₉ | CC₁₀ |
| (100 μL | control | control | GSE | GSE (50 μL = 25%)⁴ + |
| of inoculum) | M⁵ | B⁶ | (100 μL = 50%)⁴ | LiF (50 μL = 25%)⁴ |
| Staphylococcus | − | + | 50% | 12.5% |
| aureus | | | 100 μL | 25 μL |
| ATCC 29213 | | | | |
| MRSA | − | + | 25% | 6.25% |
| | | | 50 μL | 12.5 μL |
| Pseudomonas | − | + | >50% | 6.25% |
| aeruginosa | | | >100 μL | 12.5 μL |
| ATCC 27853 | | | | |
| Escherichia | − | + | >50% | 25% |
| coli | | | >100 μL | 50 μL |
| ATCC 25922 | | | | |
| Streptococcus | − | + | 12.5% | 6.25% |
| pneumoniae | | | 25 μL | 12.5 μL |
| ATCC 49619 | | | | |

| | MIC (% v/v and μL¹) of the bacterial strains | | |
|---|---|---|---|
| | by GSE alone or combined with LiF (8 mg/L)², or | | |
| | with LiF (8 mg/L)² + Disper ®³, or | | |
| | with LiF (8 mg/L)² + SnF₂ (5 mg/L)² + Disper ®³, or | | |
| | with LiF (8 mg/L)² + CoF₂ (4 mg/L)² + Disper ®³ | | |
| | | C₂₆ | C₂₇ |
| Bacterial | C₂₅ | GSE (3.33 μL = 1.66%)⁴ + | GSE (3.33 μL = 1.66%)⁴ + |
| strains | GSE (5 μL = 2.5%)⁴ + | LiF (3.33 μL = 1.66%)⁴ + | LiF (3.33 μL = 1.66%)⁴ + |
| (100 μL | LiF (5 μL = 2.5%)⁴ + | SnF₂ (3.33 μL = 1.66%)⁴ + | CoF₂ (3.33 μL = 1.66%)⁴ + |
| of inoculum) | Disper ® (90 μL = 45%)⁴ | Disper ® (90 μL = 45%)⁴ | Disper ® (90 μL = 45%)⁴ |
| Staphylococcus | 0.625% | 0.4% | 0.4% |
| aureus | 1.25 μL | 0.8 μL | 0.8 μL |
| ATCC 29213 | | | |
| MRSA | 0.31% | 0.2% | 0.2% |
| | 0.62 μL | 0.4 μL | 0.4 μL |
| Pseudomonas | 0.625% | 0.4% | 0.4% |
| aeruginosa | 1.25 μL | 0.8 μL | 0.8 μL |
| ATCC 27853 | | | |
| Escherichia | 2.5% | 0.8% | 0.8% |
| coli | 5 μL | 1.6 μL | 1.6 μL |
| ATCC 25922 | | | |
| Streptococcus | 0.625% | 0.4% | 0.4% |
| pneumoniae | 1.25 μL | 0.8 μL | 0.8 μL |
| ATCC 49619 | | | |

¹μL per 100 μL of inoculum.
²End concentrations of LiF, SnF₂ and CoF₂ in the medium, i.e. 1.6 μg of LiF or 1.6 μg of LiF + 1 μg of SnF₂ or 1.6 μg of LiF + 0.8 μg of CoF₂ in the 200 μL of each appropriate pit.
³[(GSE + LiF) or (GSE + LiF + SnF₂) or (GSE + LiF + CoF₂] + [Disper] [10:90].
⁴Percentage of each product in the 200 μL of each appropriate pit.
⁵Negative control (sterile culture medium): 0% bacterial growth.
⁶Positive control (inoculum): 100% bacterial growth.

As in the above examples, the experimental results presented in table 6 show that, for each microorganism, the inhibiting compositions according to the invention allow the inhibition of bacteria with doses of active ingredients much lower than that necessary when the inhibiting compositions only contain the active ingredient or the active ingredient combined with a metal activating agent (hence without dispersing agent, of liposomal type for example).

The addition of lithium fluoride to grapefruit seed extract in the composition CC₁₀ according to the prior art allows the increase of the inhibiting potency of grapefruit seed extract, the MICs of which are divided by 2 or 4 according to the microorganisms, while reducing the percentage of grapefruit seed extract used, which goes down from 50% to 25%.

The combination of the dispersing agent with lithium fluoride in the composition C₂₅ according to the invention significantly increases the efficacy of grapefruit seed extract, the MICs of which are reduced either by 95% or by 98.76%, according to the microorganisms, in comparison with those of grapefruit seed extract used alone (composition CC₉). At the same time, the percentage of grapefruit seed extract used goes down from 50% to 2.5%.

In the compositions C₂₆ and C₂₇ according to the invention, the double activation of lithium fluoride and stannous fluoride and that of lithium fluoride and cobalt fluoride allow, added to the dispersing agent, the reduction of the MICs either by 96.8%, or by 99.2%, according to the microorganisms in comparison with those of grapefruit seed extract used alone (composition CC₉). And the percentage of grapefruit seed extract used goes down from 50% to 1.66%, as in the above examples.

Therefore the compositions C₂₆ and C₂₇ according to the invention allow the division of the amount of grapefruit seed extract sufficient to inhibit these bacteria by 31.25 (for Streptococcus pneumoniae ATCC 49619) or by 62.5 (for Escherichia coli ATCC 25922) or by 125 (for Staphylococcus aureus ATCC 29213, meticillin resistant Staphylococcus aureus [MRSA] and Pseudomonas aeruginosa ATCC 27853).

1. Composition inhibiting or destroying at least one living or unicellular organism, which comprises at least one anti-infectious agent and at least one activating agent containing at least one metal element, said composition being characterized in that it further comprises at least one combined dispersing agent which is liposomal or micellar or peptidic, or in the form of microemulsion or of nanoemulsion, and
in that said metal element of the activating agent is present in said composition in an amount not exceeding 20 mg/L.

2. Composition according to claim 1, characterized in that the dispersing agent is chosen among the natural or synthetic liposomes of anionic or cationic type.

3. Composition according to any single claim above, characterized in that the activating agent comprises one unit metal element or a combination of several unit metal elements, which comes in the form of a metal colloid, or a metal salt, or in the form of a metal ion.

4. Composition according to any single claim above, characterized in that the anti-infectious agent is chosen among antibiotics, bactericides, fungicides, virucides, antiparasitics, surface disinfectants, spermicides, and phyto-sanitary compounds, whatever their chemical, vegetable or natural origin.

5. Composition according to claim 4, characterized in that the anti-infectious agent is an antibiotic chosen among penicillins, cephalosporins, cyclines, aminosides, macrolides, sulfamides, quinolones, and phenicolated antibiotics.

6. Composition according to claim 4, characterized in that the anti-infectious agent is a chemical virucide chosen among quaternary ammonium compounds, or a virucide of vegetable origin chosen among the essential oils of oregano, thyme, savory or among plant extracts, or a virucide of natural origin.

7. Composition according to claim 4, characterized in that the anti-infectious agent is a chemical bactericide chosen among quaternary ammonium compounds, biguanides, carbanilides, phenolic compounds, chlorinated compounds, and glutaraldehyde.

8. Composition according to claim 6 or 7, characterized in that the anti-infectious agent is tetradecyl-dimethyl-benzyl-ammonium fluoride, and said composition may be administered by injection.

9. Composition according to claim 4, characterized in that the anti-infectious agent is a spermicide chosen among quaternary ammonium compounds, p-menthanylphenyl polyoxyethylene ether and nonoxynol 9.

10. Composition according to claim 4, characterized in that the anti-infectious agent is a chemical fungicide chosen among econazole, ketoconazole, ciclopirox olamine, or a fungicide of vegetable origin chosen among the essential oils of rose geranium (Pelargonium asperum), palmarosa (Cymbopogon martinii variety motia), and spike lavender (Lavandula latifolia), or among plant extracts, or a fungicide of natural origin, and preferably propolis.

11. Composition according to claim 4, characterized in that the anti-infectious agent is a phyto-sanitary product, and preferably Neem oil.

12. Composition according to claim 4, characterized in that the anti-infectious agent is a product of vegetable origin chosen among essential oils, macerated extracts, tinctures, dry extracts, aqueous extracts, total extracts of plants, and all type of plant extract, whatever its method of extraction or of production, or a product of natural origin, and preferably propolis.

13. Utilization of the composition as it is defined according to any single claim from 1 to 12, in a cosmetic or hygiene product, insofar as it contains an anti-infectious agent, or in a phyto-sanitary product, or food preservative agents.

14. Utilization, in a composition inhibiting or destroying at least one living or unicellular organism comprising at least one anti-infectious agent, of the combination of at least one activating agent containing at least one metal element in an amount not exceeding 20 mg/L in said composition with one dispersing agent which is liposomal or micellar or peptidic, or in the form of microemulsion or of nanoemulsion, in order to increase the inhibiting or destroying potency of said anti-infectious agent.

15. Utilization of the composition according to any single claim from 1 to 12, characterized in that it allows the activation of virucides to combat viruses, the activation of bactericides to combat infectious agents of the bacterial families, the activation of fungicides to combat the appearance of fungi, and the activation of spermicides.
